(19) United States
(12) Patent Application Publication
(10) Pub. No.: US 2026/0047918 A1
(43) Pub. Date: Feb. 19, 2026

(54) TUBULAR GRAFTS AND METHODS FOR USE

(71) Applicant: THE BOARD OF TRUSTEES OF THE LELAND STANFORD JUNIOR UNIVERSITY, Stanford, CA (US)

(72) Inventors: Y. Joseph Woo, Stanford, CA (US); Yuanjia Zhu, Stanford, CA (US)

(21) Appl. No.: 19/370,600

(22) Filed: Oct. 27, 2025

Related U.S. Application Data
(63) Continuation of application No. PCT/US2024/027816, filed on May 3, 2024.
(60) Provisional application No. 63/463,820, filed on May 3, 2023.
(60) Provisional application No. 63/463,825, filed on May 3, 2023.

(51) Int. Cl. A61F 2/06

(52) U.S. Cl. CPC A61F 2/06; A61F 2230/0006; A61F 2230/0067; A61F 2230/0069; A61F 2250/0019; A61F 2250/0037; A61F 2250/0039; A61F 2250/0097

(57) ABSTRACT

Prosthetic grafts are provided that include a tubular body formed from fabric comprising an open proximal end, an open distal end, and a passage extending along a central axis between the proximal and distal ends, wherein the tubular body comprises a first section extending from the proximal end towards the distal end having a first diameter, and a second section at the distal end having a second diameter greater than the first diameter to facilitate folding the second section over an adjacent region of the first section, e.g., to provide a thicker distal end having a substantially uniform diameter. Prosthetic grafts are provided, e.g., for implantation during Ross procedures.

### RELATED APPLICATION DATA

The present application is a continuation of co-pending International Application No. PCT/US2024/027816, filed May 3, 2024, which claims benefit of U.S. provisional applications Ser. Nos. 63/463,820 and 63/463,825, both filed May 3, 2023, the entire disclosures of which are expressly incorporated by reference herein.

### STATEMENT REGARDING FEDERALLY SPONSORED RESEARCH AND DEVELOPMENT

None.

### TECHNICAL FIELD

The present application relates to medical devices and, more particularly, to prosthetic tubular grafts, e.g., for replacing a segment of an aorta or other blood vessel, and to composite inclusion grafts, e.g., for implantation within or adjacent a patient's heart during a Ross procedure, and to methods for implanting such grafts.

### BACKGROUND

Aortic replacement procedures are complex operations, oftentimes accompanied with hemostasis issues around the anastomosis suture lines. When bleeding occurs in the anterior surface of the anastomosis, surgical repair can be performed easily, but when the bleeding occurs in the posterior surface, especially regions that may be difficult to access, such as in the distal ascending aorta, aortic arch, or the aortic root, it can be extremely challenging to repair. Therefore, it is of utmost importance to perform the hemostasis suture line well. Currently on the market, aortic grafts generally have a uniform cylindrical geometry. In patients with coagulopathy or diseased aorta with severe calcification or extremely friable tissues, sewing one layer of the aortic graft to native aorta tissue may not be enough to create an effective hemostatic seal. To address this problem, some surgeons may fold one end of the aortic grafts so that two layers of the graft can be sewn to the native aorta together, which may enhance hemostasis. However, due to the cylindrical geometry, it can be difficult to fold the aortic graft and generate a smooth uniform surface for sewing.

The Ross procedure was developed to replace diseased aortic valves with autologous substitutes, namely a pulmonary autograft. However, late autograft dilation is one of the main limitations of this procedure. This dilation can lead to geometric distortion of the neo-aortic root, resulting in regurgitation. The inclusion technique was developed to support the pulmonary autograft in the aortic position and prevent this late autograft dilation. However, the inclusion technique can be technically challenging. Pulmonary valve commissure positions and orientations can greatly impact leaflet coaptation, function, and hemodynamics. Additionally, the pulmonary annulus could be easily distorted during the implantation process, leading to improper leaflet coaptation and regurgitation. This problem is especially prominent when the pulmonary autograft needs to be placed in a diseased aortic annulus, i.e., with severe annular calcification or with abnormal geometries, such as in the case of bicuspid aortic valve.

Lastly, when performing a coronary button anastomosis, some surgeons may prefer to sew the buttons directly to the pulmonary autograft without incorporating a Dacron graft, whereas other surgeons may prefer to perform a three-layered anastomosis by including the Dacron graft. However, the three-layered coronary button anastomosis is not only time-consuming to perform, but the anastomosis oftentimes is not hemostatic, leading to bleeding and dissecting between different layers of grafts and pulmonary artery tissues. This bleeding problem could be difficult to fix, and if not addressed promptly, the continued bleeding between the graft and pulmonary artery may lead to pressure built up, thereby occluding blood flow into the coronary arteries, resulting myocardial malperfusion or ischemia.

Accordingly, prosthetic grafts that facilitate replacing segments of an aorta or other blood vessel, e.g., with specialized geometry for enhanced, customizable hemostatic anastomosis suture line, and/or that facilitate replacing valves, such as an aortic valve during a Ross procedure, would be useful.

### SUMMARY

The present application is directed to medical devices and, more particularly, to prosthetic tubular grafts, e.g., for replacing a segment of an aorta or other blood vessel, and to methods for implanting such grafts. The present application is also directed to composite inclusion grafts, e.g., for implantation within or adjacent a patient's heart, e.g., during a Ross procedure, and to methods for implanting such grafts.

In accordance with one example, an aortic graft is provided that includes a distal segment having a slightly bigger diameter than the main graft segment. This way, when folding the graft down, a smooth double layered aortic graft may be generated. Optionally, the graft may include an interior skirt, e.g., in either the short or long axis direction of the graft, that may provide an extension of the aortic graft after the distal segment is folded down. Such a skirt may allow for single-layer anastomosis as is done traditionally or serve as another layer of graft material for an enhanced anastomosis suture line. Such a uniform folded graft may minimize the risk of blood leaking through the anastomosis when the graft is anastomosed to a segment of aorta or other blood vessel.

In accordance with another example, a prosthetic graft is provided that includes a tubular body formed from fabric comprising an open proximal end, an open distal end, and a passage extending along a central axis between the proximal and distal ends, wherein the tubular body comprises a first section extending from the proximal end towards the distal end having a first diameter and an axial first length, and a second section at the distal end having a second diameter greater than the first diameter to facilitate folding the second section over an adjacent region of the first section, the second section having an axial second length shorter than the first length.

In accordance with still another example, a prosthetic graft is provided that includes a tubular body formed from fabric comprising an open proximal end, an open distal end, and a passage extending along a central axis between the proximal and distal ends, wherein the tubular body comprises a first section extending from the proximal end towards the distal end having a substantially uniform first diameter and an axial first length, a second section at the distal end having a substantially uniform second diameter greater than the first diameter, and a transition between the first and second sections to facilitate folding the second section over an adjacent region of the first section, the second section having an axial second length shorter than the first length; and an annular skirt attached to the graft adjacent the second section.

In accordance with yet another example, a prosthetic graft is provided that consists of a tubular body formed from fabric comprising an open proximal end, an open distal end, and a passage extending along a central axis between the proximal and distal ends, wherein the tubular body comprises a first section extending from the proximal end towards the distal end having a substantially uniform first diameter and an axial first length, a second section at the distal end having a substantially uniform second diameter greater than the first diameter, and a transition between the first and second sections to facilitate folding the second section over an adjacent region of the first section, the second section having an axial second length shorter than the first length.

In accordance with still another example, a prosthetic graft is provided that consists of a tubular body formed from fabric comprising an open proximal end, an open distal end, and a passage extending along a central axis between the proximal and distal ends, wherein the tubular body comprises a first section extending from the proximal end towards the distal end having a substantially uniform first diameter and an axial first length, a second section at the distal end having a substantially uniform second diameter greater than the first diameter, and a transition between the first and second sections to facilitate folding the second section over an adjacent region of the first section, the second section having an axial second length shorter than the first length; and an annular skirt attached to the graft adjacent the second section.

In accordance with another example, a method is provided for replacing a blood vessel that includes providing a prosthetic graft, comprising a tubular body formed from fabric comprising an open proximal end, an open distal end, and a passage extending along a central axis between the proximal and distal ends, wherein the tubular body comprises a first section extending from the proximal end towards the distal end having a first diameter, and a second section at the distal end having a second diameter greater than the first diameter to facilitate folding the second section over an adjacent region of the first section; removing a section of the blood vessel to create first and second vessel segments; attaching the proximal end of the graft to the first segment; folding the second section of the graft over an adjacent region of the first section; and attaching the folded second section of the graft to the second segment.

In accordance with another example, a composite inclusion graft device is provided that may be very intuitive to use, may be easy to incorporate, may prevent neo-annulus of the composite graft distortion in the aortic root, and/or may allow for different options for coronary artery button anastomosis. In one example, the device includes a substantially straight Dacron graft with a rigid sewing ring at a proximal end for autograft proximal anastomosis. In one example, the sewing ring may have a substantially circular shape lying within a plane. Alternatively, the graft may have other shapes and/or configurations, e.g., a scalloped shape or with neosinus shape similar to a Valsalva graft, a uni-graft, and the like. For example, the sewing ring may have a circular shape with two or three scallops or undulations above and below a plane around a circumference of the sewing ring. In one example, the sewing ring may include two undulations, e.g., corresponding to the shape of a native, symmetrical bicuspid aortic valve (BAV). In another example, the sewing ring may include three substantially symmetrical scallops or undulations around the circumference, e.g., corresponding to a tricuspid aortic valve, or three asymmetrical scallops or undulations, e.g., corresponding a non-symmetrical bicuspid aortic valve.

The distal end of the autograft can be anastomosed to the Dacron graft to generate the composite pulmonary autograft via the inclusion technique. This composite autograft can then be implanted into the aortic root, e.g., in similar fashion as when performing a Bentall procedure. Optionally, the graft may include markings, e.g., three-line markings that are offset from one another around the circumference of the graft, e.g., one hundred degrees (120°) away from each other, which may provide reference points for pulmonary valve commissure suspension onto the Dacron graft.

Optionally, at locations on the graft corresponding to left and right coronary sinus regions of an aortic root, one or more large circular markings may be provided. For example, two circular markings may be provided that are spaced apart circumferentially around the graft, e.g., corresponding to left and right coronary sinuses in an aortic root. Alternatively, three circular markings may be provided, e.g., including a third circular marking aligned with the non-coronary artery sinus. The markings may facilitate performing direct anastomosis of coronary artery buttons to the pulmonary artery, e.g., the two circular areas may be easily cut out to allow the coronary artery buttons to be sutured to the graft. Alternatively, to perform a three-layered anastomosis, one may proceed with the anastomosis in the usual fashion without cutting out the two circular areas.

In accordance with one example, prosthetic graft is provided that includes a tubular body formed from fabric comprising an open proximal end, an open distal end, and a passage extending along a central axis between the proximal and distal ends; a sewing cuff extending around the perimeter of the proximal end; and a plurality of linear markings spaced apart from one another around a circumference of the tubular body, each linear marking extending along a wall of the tubular body at least partially between the proximal and distal ends. Optionally, graft may include one or two enclosed markings surrounding respective regions on the wall of the tubular wall to facilitate performing an anastomosis at the regions.

In accordance with another example, a prosthetic graft is provided that includes a tubular body formed from fabric comprising an open proximal end, an open distal end, and a passage extending along a central axis between the proximal and distal ends; a sewing cuff extending around the perimeter of the proximal end; and one or more enclosed markings, each marking surrounding a region on the wall of the graft to facilitate performing an anastomosis at the region. Optionally, the graft may include one, two, or three enclosed markings surrounding respective regions on the wall of the tubular wall to facilitate performing an anastomosis at the regions, e.g., spaced apart around a circumference of the graft. Optionally, the graft may include a prosthetic valve, e.g., a pulmonary valve autograft, attached within the graft, e.g., within the proximal end adjacent the sewing cuff.

In accordance with still another example, a method is provided for replacing an aortic valve within a heart of a patient that includes providing a prosthetic graft including a tubular body formed from fabric comprising an open proximal end, an open distal end, and a passage extending along a central axis between the proximal and distal ends, a sewing cuff extending around the perimeter of the proximal end, and a plurality of linear markings spaced apart from one another around a circumference of the tubular body; attaching a prosthetic valve to the prosthetic graft to provide a composite inclusion graft; removing the aortic root adjacent a valve annulus of the aortic valve to create a separated end of the aorta; excising the native aortic valve from the valve annulus; introducing the composite inclusion graft such that the linear markings are aligned with commissures of the valve annulus; securing the sewing cuff adjacent the valve annulus; and securing the distal end of the tubular body to the separated end of the aorta.

In accordance with yet another example, a method is provided for replacing an aortic valve within a heart of a patient that includes providing a prosthetic graft including a tubular body formed from fabric comprising an open proximal end, an open distal end, and a passage extending along a central axis between the proximal and distal ends, a sewing cuff extending around the perimeter of the proximal end, and a plurality of linear markings spaced apart from one another around a circumference of the tubular body; removing the aortic root adjacent a valve annulus of the aortic valve; excising the native aortic valve from the valve annulus; introducing the graft such that the linear markings are aligned with commissures of the valve annulus; securing the sewing cuff adjacent the valve annulus; securing the distal end of the tubular body to the end of the remaining aorta; and implanting a prosthetic valve within the graft adjacent the valve annulus.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIGS. 1A and 1B show an example of a tubular graft including a relatively larger diameter distal end.

FIG. 2 is a cross-section of the tubular graft of FIGS. 1A and 1B with the distal end folded back over the adjacent graft material.

FIG. 3 is a cross-section of another example of a tubular graft including a relatively larger diameter distal end and a skirt extending axially within the distal end.

FIGS. 4A and 4B show an example of a tubular graft for a composite inclusion graft.

FIGS. 5A and 5B show another example of a tubular graft for a composite inclusion graft.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

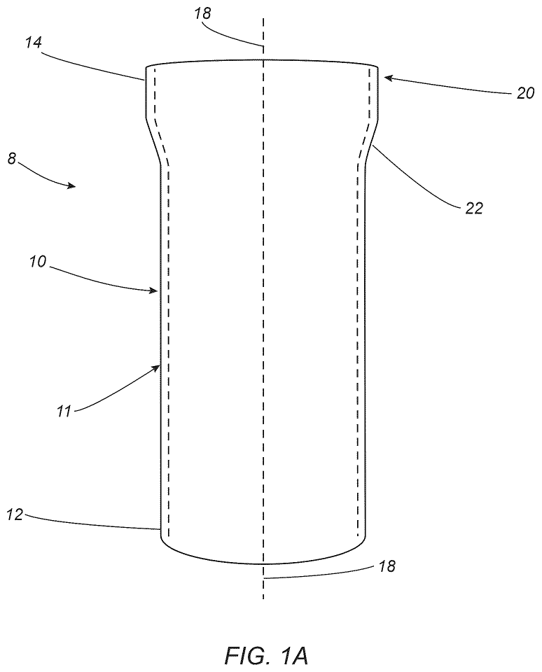

Turning to the drawings, FIGS. 1A and 1B show an example of a prosthetic tubular graft device 8 that generally includes a tubular graft or body 10 including an open proximal or inlet end 12, an open distal or outlet end 14, and a passage 16 extending along a central axis 18 between the proximal and distal ends 12, 14.

Along most of its length, the graft 10 may include a first or main section 11 having a substantially uniform first diameter, e.g., corresponding to the diameter of a segment of an aorta or other blood vessel being replaced, e.g., between about twenty and three hundred sixty millimeters (20-360 mm). In one example, the graft 10 may have an overall length between the proximal and distal ends 12, 14 sufficient to reconnect end segments of an excised vessel, e.g., between about fifty and three hundred centimeters (50-300 cm).

At one end, e.g., the distal end 14 as shown, the graft 10 may include a second or end section 20 having a second diameter slightly larger than the first section 11. For example, as shown, the second section 20 may have a length that is substantially shorter than the first section 11, e.g., between about five and twenty millimeters (5.0-20 mm) and a substantially uniform diameter larger than the first section 11. For example, the second diameter may be slightly larger than the first diameter and the thickness of the graft material.

In addition, the graft 10 may include a transition region 22 between the sections 11, 20, e.g., having a frustoconical shape as shown, or other tapered or flared shape extending between the first section 11 and the second section 20.

The graft 10 may be constructed from conventional materials, e.g., fabric such as polyester (e.g., Dacron) and/or other biocompatible material. For example, the first and second sections 11, 20 may be formed from a single tubular sleeve of fabric, e.g., having similar wall thickness and/or other construction other than the different diameters.

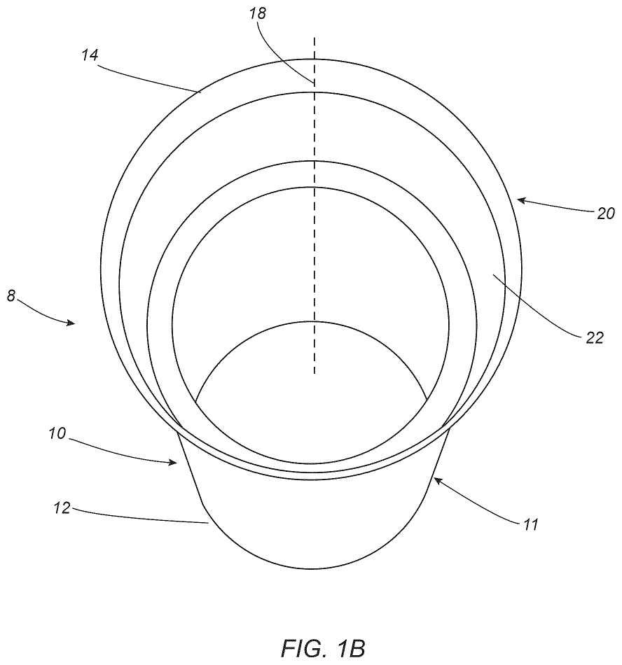

The difference in diameters may facilitate folding the second section 20 back over an adjacent region of the first section 11 without risk of the graft 10 bunching, kinking, or otherwise impacting the uniformity of the lumen 16. For example, as shown in FIG. 2 and as described elsewhere herein, the second section 20 may be folded back over the adjacent region of the first section 11 while maintaining a substantially uniform cylindrical lumen along the entire length of the graft 10.

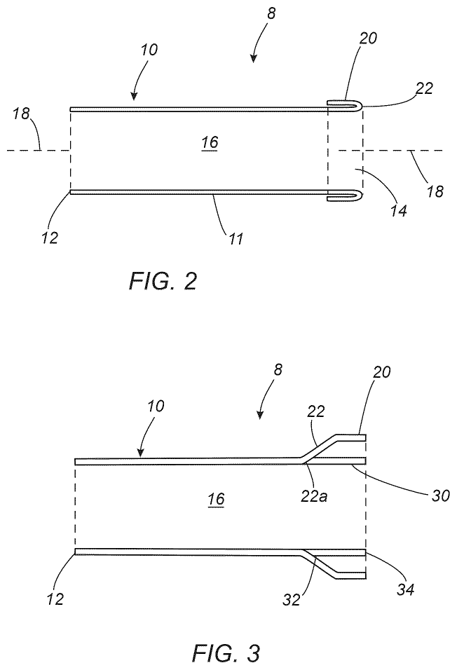

Optionally, as shown in phantom in FIG. 3, the graft device 8′ may include an annular skirt 30 that is disposed within the second section 20 of the graft 10, e.g., that extends from the smaller end 22a of the transition 22, i.e., at the end of the first section 11, towards the distal end 14 of the graft 10. As shown, the skirt 30 may have an axial length shorter than the second section 20 or, alternatively, the skirt 30 may be longer than the second section 20, e.g., such that the skirt 30 extends beyond the distal end 14 of the graft 20 (not shown).

The skirt 30 may be formed materials similar to the graft 10, e.g., fabric, with a first end 32 of the skirt 30 attached to the graft 10, e.g., by one or more of suturing, bonding with adhesives, fusing, sonic welding, and the like, such that the skirt 30 is aligned along the central axis 18, with a second end 34 of the skirt 30 remaining free within the second section 11. Thus, when the second section 20 is folded back over the first section 11, the skirt 30 may provide a tubular extension having an inner diameter similar to the first section 11 of the graft 10.

Optionally, the graft 10 may include one or more reinforcements if desired, e.g., annular stents spaced apart from one another along the length of the graft, axial struts that extend at least partially between the proximal and distal ends, and the like (not shown). For example, such reinforcements may be attached around the exterior of the graft 10, e.g., using one or more sutures, bonding with adhesive, fusing, sonic welding, and the like, e.g. to support the graft 10 from collapsing inwardly.

A set of devices similar to the graft device 8 may be provided with a variety of lengths of the first section 11, e.g., such that an appropriate length device 8 may be selected during a surgical procedure, e.g., to replace a section of an aorta or other blood vessel. Alternatively, the proximal end 12 of the graft 10 may be cut or otherwise separated during a procedure to provide a desired length. In a further alternative, if desired, a larger diameter section may be provided on both ends of the graft, with or without a skirt (not shown), e.g., if it is desired to fold both ends during a procedure.

The graft device 8 may be used to replace or repair a section of a blood vessel, such as a section of a patient's aorta including one or more of the ascending aorta, aortic arch, or the aortic root. After the desired portion of the aorta is removed, the graft device 8 may be connected to the resulting first and second end segments, e.g., by suturing and/or other anastomosis. For example, the proximal end 12 of the graft 10 may be sewn or otherwise connected to the first segment, e.g., the more anterior segment, which may be more easily accessed and/or visually observed. For the second segment, e.g., the more posterior segment, which may not be as easily accessed, the distal end 14 of the graft 10 may be sewn or otherwise connected to the segment after folding the second section 11 of the graft 10, as shown in FIG. 2. As described elsewhere herein, the folded end may facilitate performing an anastomosis on more difficult to reach and/or observe segments, providing additional fabric to receive sutures, thereby minimizing the risk of leaks at the second anastomosis, while maintaining a substantially uniform diameter in the graft 10.

If, as shown in FIG. 3, the device 8′ includes a skirt 30 concentric with the second section 11, the skirt 30 may be positioned over the posterior aorta segment and, if desired, additional sutures may be applied through the skirt 30 into the aorta to further enhance sealing. Alternatively, the skirt 30 may be used to perform the anastomosis at the distal end 14 and the folded section may not need to receive sutures.

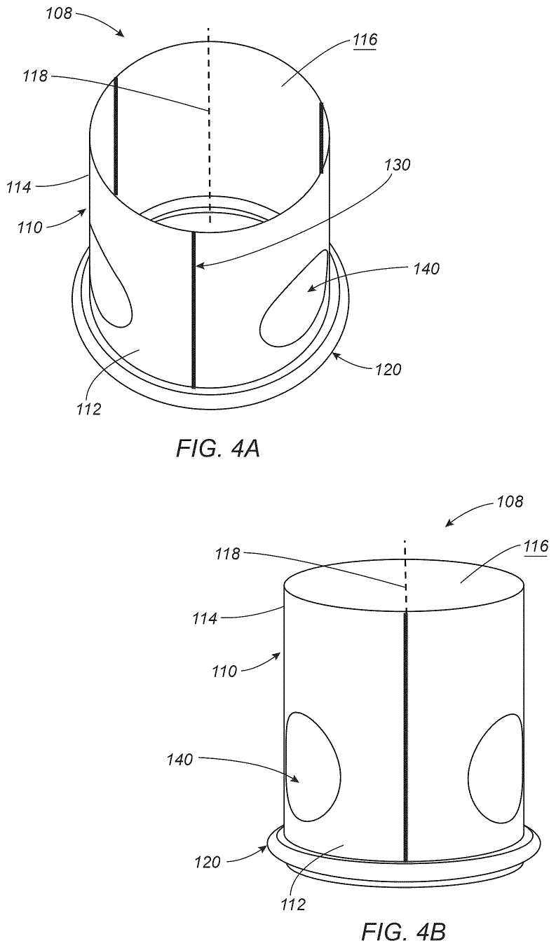

Turning to FIGS. 4A and 4B, an example of a prosthetic tubular graft device 108 is shown that generally includes a tubular graft or body 110 including an open proximal or inlet end 112, an open distal or outlet end 114, and a passage 116 extending along a central axis 118 between the proximal and distal ends 112, 114. The graft 110 may be constructed from conventional materials, e.g., fabric such as polyester (e.g., Dacron) and/or other biocompatible material.

A sewing ring or cuff 120 may be attached to the graft 110, e.g., around the proximal end 112, which may be constructed from biocompatible materials, such as polyester (e.g., Dacron). The sewing cuff 120 may include multiple layers of fabric or may be filled with foam, fabric, and/or other material, which may be expandable or non-expandable, to facilitate securing the sewing cuff 120 within or adjacent a native valve annulus (not shown). The sewing cuff 120 may be permanently attached to the proximal end 112 of the graft 110, e.g., by one or more of sutures, bonding with adhesive, fusing, interference fit, one or more connectors, and the like. The sewing cuff 120 may be substantially rigid, e.g., having a circular outer perimeter, or, alternatively, a multiple lobed or scalloped outer perimeter, e.g., including three lobes similar to an aortic valve annulus, a generally “D” shaped perimeter, e.g., similar to a mitral valve annulus, or other shape (not shown), similar to the shape of the native annulus into which the prosthetic valve is to be implanted.

The sewing cuff 120 may have uniform rigidity about the perimeter or, alternatively, different regions of the sewing cuff 120 may have varying rigidity, e.g., rigid regions separated by semi-rigid or more flexible regions. Alternatively, the sewing cuff 120 may be biased to an expanded circular or other shape, but may be compressed into a contracted condition, e.g., to facilitate introduction into the valve annulus, whereupon the sewing cuff 120 may automatically expand when released, e.g., to engage, dilate, and/or otherwise shape the native valve annulus.

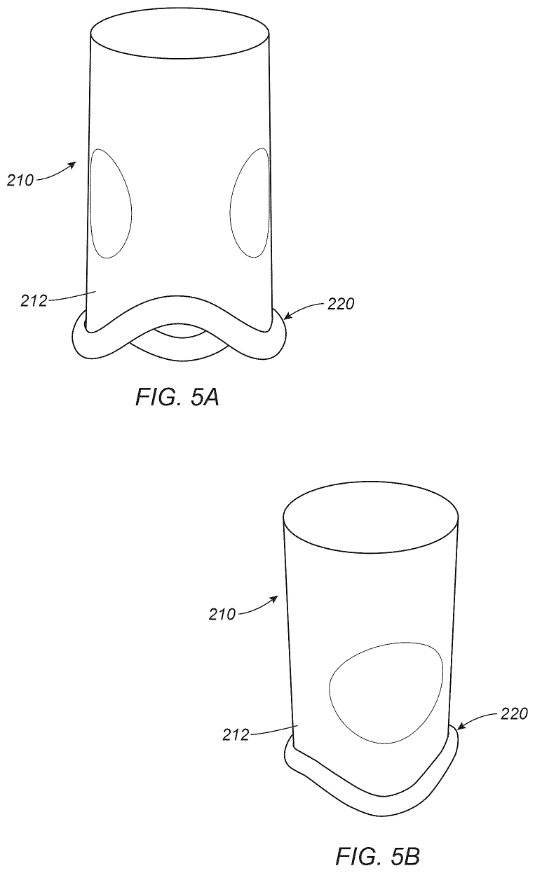

The sewing cuff 120 may lie entirely within a plane, e.g., as shown in FIGS. 4A and 4B. Alternatively, as shown in FIGS. 5A and 5B, a sewing cuff 220 may be provided on a graft 210 (otherwise similar to the graft 110) that has an undulating shape around its circumference, e.g., extending above and/or below a plane. For example, the sewing cuff 220 (and the proximal end of the graft 210) may include three undulations around its circumference, e.g., similar to the profile of a native aortic valve annulus, which may substantially symmetrical around the circumference or arranged asymmetrically if desired. Alternatively, the sewing ring may have a circular shape including two undulations (not shown), e.g., corresponding to the shape of a native, symmetrical bicuspid aortic valve (BAV).

Optionally, with continued reference to FIGS. 4A and 4B, the graft 110 may include a plurality of markings around its circumference, e.g., a plurality of linear markings extending at least partially between the proximal and distal ends 112, 114, e.g., substantially parallel to the central axis 118. The linear markings may provide reference points for valve commissure attachments in the graft. For example, as shown, three straight linear markings 130 may be provided that are offset from one another around the circumference of the graft, e.g., one hundred degrees (120°) away from each other, which may provide reference points for pulmonary valve commissure suspension onto the graft 110. The linear markings 130 may be any desired color, e.g., black, and/or may be continuous or discontinuous, e.g., dashed, dotted, and the like (not shown), which may facilitate observing the markings within a surgical field. The linear markings 130 may be provided on the outer surface of the graft 10, the inner surface, or both, e.g., as shown. Although three markings are shown, the graft may include any number of markings, e.g., two linear markings spaced apart about one hundred eighty degrees (180°) from one another, four or more markings, and the like (not shown).

In addition, the graft 110 may include one or more enclosed markings, e.g., to facilitate performing an anastomosis to one or more blood vessels. For example, as shown, a pair of circular markings 140 may be provided that are offset from one another around the circumference of the graft 110. In the example shown, the circular markings 140 are offset nonuniformly around the circumference, e.g., spaced apart about one third of the circumference on one side of the graft 110 and about two thirds of the circumference on the other side of the graft 110, e.g., such that the circular markings 140 are located at locations approximating the locations of coronary arteries relative to an aortic root. Alternatively, only one circular marking may be provided, or three circular markings may be provided, e.g., spaced apart about the circumference (not shown). In a further alternative, the enclosing markings 140 may be spaced at different locations around the circumference and/or along the length of the graft 110 from one another and/or any desired number of markings may be provided, as desired.

The circular markings 140 may be any desired color, e.g., black, and/or may be continuous or discontinuous, e.g., dashed, dotted, and the like (not shown), which may facilitate observing the markings within a surgical field. Optionally, the circular markings 140 may include weakened perimeters to facilitate removing portions of the graft 110 encompassing the circular markings 140. For example, a series of perforations may be provided around the perimeters of the markings that are spaced apart from one another or the material may be scored such the perimeter is thinner than the adjacent material to facilitate separation.

During use, the graft device 108, together with an implanted autograft such as a composite inclusion graft, may be implanted adjacent or within a native valve annulus, such as the aortic valve annulus (not shown), e.g., after removing the native valve. After aortic cross-clamp, the site of the native valve annulus may be accessed, e.g., using sternotomy or using minimally invasive approaches.

For example, the native aortic root, e.g., including a desired length of the descending aorta, and the aortic valve may be surgically excised, and the coronary arteries severed. The proximal and distal ends 112, 114 of the graft 110 may then be secured between the aortic valve annulus and the remaining end of the aorta, e.g., using one or more sutures, clips or other fasteners, and the like. For example, the sewing cuff 120 may be sutured within or adjacent the remaining native aortic valve annulus and the distal end 114 of the graft 110 may be sutured to the aorta immediately adjacent the excised segment. The sewing cuff 120 may engage the surrounding tissue of the native annulus, e.g., to enhance sealing, and/or may dilate or otherwise shape the native annulus, if desired.

For example, during a Ross procedure, the patient's pulmonary valve may be surgically removed using conventional methods, and the resulting autograft may be attached within the graft 110, e.g., which will be ultimately implanted within the native aortic valve annulus, using sutures and/or other conventional methods. The annulus of the pulmonary autograft may be positioned within the sewing cuff 120 and attached to the graft 110 to provide a composite inclusion graft 108. For example, one or more sutures may be used to perform an anastomosis to attach the autograft within the sewing cuff 120, e.g., to support and/or stabilize the pulmonary annulus during implantation. The patient's pulmonary valve may be replaced by a homograft, a prosthetic valve, e.g., a xenograft including a tissue annulus carrying tissue leaflets similar to the excised pulmonary valve, a mechanical valve, a bioprosthetic valve including leaflets constructed from pericardium or other tissue, or other prosthetic valve.

The linear markings 130 may facilitate visually orienting the autograft during introduction and suturing within the graft 110, e.g., after implanting the graft 110. For example, during introduction of the graft 110, the linear markings 130 and/or circular markings 140 may be oriented, e.g., by rotating the graft 110 such that the linear markings 130 are aligned with the commissures adjacent the native annulus and/or the circular markings 140 may be aligned with the respective coronary arteries of the patient. Thereafter, the commissures of the autograft may then be aligned with the linear markings 130 during introduction to align the autograft commissures with the commissures of the native annulus and/or to facilitate identifying locations for the sutures or other fasteners used to secure the autograft. Alternatively, the commissures of an autograft pulmonary valve may be aligned with the markings 130 during introduction and subsequent attachment to the graft 110 before introducing the assembled graft 108 into the native annulus.

The sewing cuff 120 may then be secured to the native valve annulus, e.g., by directing one or more sutures through the sewing cuff 120 and the surrounding tissue, thereby securing the pulmonary autograft relative to the native annulus. The rigidity of the sewing cuff 120 may prevent distortion of the pulmonary annulus during implantation, particularly within a diseased and/or abnormal aortic valve annulus. The distal end 114 of the graft 110 may then be secured to the end of the remaining aorta above the native annulus, e.g., using one or more sutures.

The coronary arteries may be attached to the autograft and/or graft. For example, when the aortic root is removed, coronary buttons may be formed using conventional methods, which may then be attached to the graft 110 and/or autograft. For example, the material around circular markings 140 may be removed, e.g., cut or otherwise separated from the surrounding graft material, and the coronary buttons may be sutured or otherwise secured to the autograft through the resulting openings. In addition, or alternatively, the coronary buttons may be sutured directly to the graft 110 around the openings. Alternatively, a three-layered anastomosis may be formed without removing the material within the circular markings.

The graft 110 may enhance providing a seal around the implanted pulmonary valve autograft and/or facilitate shaping the native annulus, e.g., to reduce the risk of leakage after implantation. Once fully implanted, the procedure may be completed using conventional methods.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A prosthetic graft, comprising:
a tubular body formed from fabric comprising an open proximal end, an open distal end, and a passage extending along a central axis between the proximal and distal ends,
wherein the tubular body comprises a first section extending from the proximal end towards the distal end having a first diameter and an axial first length, and a second section at the distal end having a second diameter greater than the first diameter to facilitate folding the second section over an adjacent region of the first section, the second section having an axial second length shorter than the first length.

2. The prosthetic graft of claim 1, wherein the tubular body comprises a transition between the first section and the second section.

3. The prosthetic graft of claim 2, wherein the transition has one of a frustoconical shape, a flared shape, and a tapered shape.

4-6. (canceled)

7. The prosthetic graft of claim 1, wherein the first length is between about fifty and three hundred centimeters (50-300 cm) and wherein the second length is between about five and twenty millimeters (5.0-20 mm).

8-10. (canceled)

11. The prosthetic graft of claim 1, further comprising an annular skirt attached to the graft adjacent the second section.

12-40. (canceled)

41. A prosthetic graft, comprising:
a tubular body formed from fabric comprising an open proximal end, an open distal end, and a passage extending along a central axis between the proximal and distal ends;
a sewing cuff extending around the perimeter of the proximal end; and
a plurality of linear markings spaced apart from one another around a circumference of the tubular body, each linear marking extending along a wall of the tubular body at least partially between the proximal and distal ends.

42. The prosthetic graft of claim 41, further comprising one or more enclosed markings, each marking surrounding a region on the wall of the graft to facilitate performing an anastomosis at the region.

43. The prosthetic graft of claim 41, wherein the one or more enclosed markings comprise a single circular marking on the wall of the graft.

44. The prosthetic graft of claim 41, wherein the one or more enclosed markings comprise two enclosed markings surrounding respective regions on the wall of the tubular wall to facilitate performing an anastomosis at the regions.

45. The prosthetic graft of claim 41, wherein the one or more enclosed markings comprise three enclosed markings surrounding respective regions on the wall of the tubular wall to facilitate performing an anastomosis at the regions.

46. The prosthetic graft of claim 44, wherein the enclosed markings are spaced apart from one another around a circumference of the tubular body.

47. The prosthetic graft of claim wherein the enclosed markings are spaced apart from one another at locations approximating locations of coronary arteries relative to an aortic root.

48. (canceled)

49. The prosthetic graft of claim 43, wherein each enclosed marking comprises a weakened region of the wall to facilitate removal of material within the enclosed marking.

50. (canceled)

51. The prosthetic graft of claim 41, wherein the linear markings are spaced apart from one another around the circumference of the tubular body.

52. The prosthetic graft of claim 51, wherein the linear markings comprise three linear markings spaced apart about one hundred degrees (120°) away from each other around the circumference.

53. The prosthetic graft of claim 41, wherein the linear markings extend an entire distance between the proximal and distal ends.

54. The prosthetic graft of claim 41, wherein the linear markings are on both an inner surface and an outer surface of the tubular body.

55. The prosthetic graft of claim 41, wherein the sewing cuff is substantially rigid.

56. The prosthetic graft of claim 41, wherein the sewing cuff is biased to an expanded condition but may be compressed to a contracted condition to facilitate introduction into the native annulus.

57-81. (canceled)

82. A method for replacing an aortic valve within a heart of a patient, comprising:
providing a prosthetic graft including a tubular body formed from fabric comprising an open proximal end, an open distal end, and a passage extending along a central axis between the proximal and distal ends, a sewing cuff extending around the perimeter of the proximal end, and a plurality of linear markings spaced apart from one another around a circumference of the tubular body;
attaching a prosthetic valve to the prosthetic graft to provide a composite inclusion graft;
removing the aortic root adjacent a valve annulus of the aortic valve to create a separated end of the aorta;
excising the native aortic valve from the valve annulus;
introducing the composite inclusion graft such that the linear markings are aligned with commissures of the valve annulus;
securing the sewing cuff adjacent the valve annulus; and
securing the distal end of the tubular body to the separated end of the aorta.

83-103. (canceled)
